(19) United States
(12) Patent Application Publication
(10) Pub. No.: US 2008/0227873 A1
(43) Pub. Date: Sep. 18, 2008

(54) Gelation of Undenatured Proteins with Polysaccharides

(76) Inventors: Sandra I. Laneuville Ballester, Quebec (CA); Sylvie L. Turgeon, Quebec (CA); Christian Sanchez, Nancy (FR); Paul Paquin, Quebec (CA)

Correspondence Address: CHOATE, HALL & STEWART LLP, TWO INTERNATIONAL PLACE, BOSTON, MA, 02110, US

(21) Appl. No.: 11/997,811

(22) PCT Filed: Aug. 4, 2005
(86) PCT No.: PCT/CA05/01216

§ 371 Date: Feb. 4, 2008

(51) Int. Cl. C07K 17/04

(52) U.S. Cl. 516/105

(57) ABSTRACT

The present invention concerns the gelation of an undenatured protein and a polysaccharide dispersions by a gradual pH adjustment of their mixture The final pH of the mixture is close to or lower than the isoelectric point of the protein when anionic polysaccharides are used. In the present invention, the undenatured protein and the polysaccharide in the gel state have net opposite electric charges. The concentration of protein and polysaccharide preferably ranges from 0.02 to 10 wt %. The present invention also concerns a gel obtained by this method.

### FIELD OF THE INVENTION

The present invention relates to the field of gelation and more specifically to a method for gelation of an undenatured protein and a polysaccharide. The present invention also relates to gels obtained by the method of the invention.

### DESCRIPTION OF THE PRIOR ART

Nowadays, less and less ingredients are introduced into the market each year due to the high costs and long time required to test their safety and be approved as food or pharmaceutical ingredients by the relevant authorities. New ways are thus necessary to increase the range of ingredients available in the food or pharmaceutical industry. One of these ways is to extend the potential uses and application of well-known and accepted molecules, such as many proteins and polysaccharides, by controlling their intermolecular interactions.

The renewed interest in the field of protein-polysaccharide interactions has been fueled by the potential and practical implications for numerous fields such as the biomedical (gene therapy, enzyme immobilization, protein recovery and purification); pharmaceutical (encapsulation, drug delivering systems); cosmetics (microencapsulation of active ingredients); and the food industry (texturing and stabilizing ingredients, flavor/ingredient encapsulation). Several reviews on protein-polysaccharide applications have been published (Renard et al. (2002); Schmitt et al. (1998); Dumitriu and Chornet (1998) and Tolstoguzov (1997)).

The mixture of proteins and polysaccharides in aqueous dispersion is often accompanied by phase separation either segregative (thermodynamic incompatibility) or associative (thermodynamic compatibility) depending mainly on the electrical charges on the biopolymers and therefore on the factors affecting them such as the ionic strength and pH (Tolstoguzov (2003); Mattison et al. (1999)). Therefore, controlling environmental factors results in the diversification of their solubility, co-solubility, mechanical, texturing, and gelation properties as well as in their behavior at interfaces (Dickinson (2003); Tolstoguzov (1997); Samant et al. (1993)).

Usually, the attractive interaction between oppositely charged biopolymers tends to produce electrostatic complexes or coacervates instead of gels. These particulated complexes have been extensively studied for applications in the food industry (Tolstoguzov (2003); Girard et al. (2002); Dickinson (1998); Dickinson and McClements (1996); Samant et al. (1993), Stainsby (1980)) and pharmaceutical industry for the production of drug delivering systems (Renard et al. (2002); Gombotz and Wee (1998); Dumitriu and Chornet (1998); Tabata and Ikada (1998)).

There has also been an extensive research in the area of protein-polysaccharide gelation under thermodynamic incompatibility conditions (Tolstoguzov (2003); Turgeon and Beaulieu (2001); Bryant and McClements (2000); Samant et al. (1993), where electrostatic repulsion forces between unlike species leads to a segregation of similar molecules in two different phases, resulting in an increased concentration in each separated phase and thus gelation can be achieved at lower concentrations than that usually needed for the gelation of the constituents alone. The concentration needed to achieve gelation in protein-polysaccharide systems under thermodynamic incompatibility conditions can be lowered from the concentrations normally used for protein gelation alone, in the range of 10-14 wt % (Kavanagh et al. (2000); Sanchez et al. (1997)), to concentrations of 6.0-8.5 wt % (e.g., Baeza et al. (2003); Olsson et al. (2002); Bryant and McClements (2000)). However, protein must still need to undergo a denaturation process through thermal or partial hydrolysis treatment for gelation to occur.

Drug delivering matrices based on biomacromolecules such as proteins and polysaccharides can be enzymatically biodegraded in the body with time (Tabata and Ikada (1998)), and accordingly several studies report the use of protein-polysaccharides microparticles (Edman et al. (1980); Ho et al. (1995)) as drug delivering systems. Numerous pharmaceutical studies have also dealt with the development of carrier gelified matrices or hydrogels, some of which require the use of cross-linking agents (Berger et al. (2004); Hennink and van Nostrum (2002); Tabata and Ikada (1998); Chen et al. (1995)) that may present different degrees of toxicity (Hennick and van Nostrum (2002)). However, one of the most important problems encountered in drug delivery systems is the loss of proteins' biological activity due to denaturation. The activity loss is principally caused by the harsh conditions encountered during the production of the delivering matrices such as heating and sonication or the treatments applied for the cross-linking agent to activate e.g., irradiation (Tabata and Ikada (1998)). No gelifying matrices based on natural biopolymers have been reported without the application of a denaturing treatment to allow protein gelation.

Finally studies have been made to improve the gelation properties of proteins by limited proteolysis or by applying a heat pre-denaturation treatment of the proteins (Foegeding et al. (2002); Britten and Giroux (2001)); these allow to subsequently achieve gelation at lower temperatures and concentrations than that required for native protein gelation.

For instance, Eissa et al. (2004) describe the gelation in an acidic medium of whey protein at a concentration of 7.5 wt %. The first step of this procedure is an enzymatic treatment of the protein, including a heat treatment at 50° C.; the second step is acidification at 25° C. until pH 4 by addition of glucono-δ-lactone.

US 2004/0091540 A1 (Desrosiers et al.) discloses an injectable solution of a gel comprising from 0.1 to 5 wt % of cellulose, a polysaccharide, polypeptide or a derivative or any mixture thereof, and 1 wt % to 20 wt % of a salt of polyol or sugar. The mixture has a pH between 6.5 and 7, gelation takes place between 4° C. and 70° C. by thermogelling and through covalent interaction.

US 200410146564 A1 (Subirade et al.) teaches the cold gelation of whey protein by addition of Ca²⁺ to a preheated protein suspension.

Alting et al. (2004 and 2002) respectively, teach the gelation of whey protein and ovalbumin in two steps. The first step consists in protein denaturation at high temperature, followed by gelation at room temperature by slow acidification with glucono-δ-lactone.

Veerman et al. (2003) teach the cold gelation of β-lactoglobulin at low concentration in presence of Ca²⁺. The procedure consists of fibrils formation at pH 2 and at high temperature, cooling the fibrils in ice, adjusting the pH to 7 or 8, and finally cross-linking of the fibrils in the presence of CaCl₂.

Remondetto and Subirade (2003) teach the cold gelation of β-lactoglobulin in presence of Fe²⁺ but in the absence of polysaccharide. The concentration of β-lactoglobulin used was 9.5%; the protein was pre-heated to 80° C. then cooled to 24° C.

Finally, US 2003/0124189 A1 (Zentner et al.) teaches the formation of an hydrogel from polymeric mixtures such as chitosan and polyether glycol in an acidic medium to regulate the delivery of bioactive ingredients. However these polymers are not cross-linked in a covalent or ionic way but are simply physically mixed.

Therefore, there is a need for new methods for gelation of undenatured proteins and polysaccharides.

### SUMMARY OF THE INVENTION

An object of the present invention is to provide a method and a gel that satisfy the above-mentioned need.

More specifically, the object of the present invention is achieved by a method for gelation of an undenatured protein and a polysaccharide, said method comprising the steps of:
a—providing a mixture of a dispersion of an undenatured protein and a dispersion of a polysaccharide;
b—stirring the mixture to obtain a homogeneous mixture;
c—gradually modifying the electronic charge of said undenatured protein and/or said polysaccharide to obtain a mixture wherein said undenatured protein and said polysaccharide have opposite charges; and
d—resting the mixture of step c) for a period of time suitable to form a gel.

The present invention also relates to a gel obtained by the method according to the invention.

The main advantage of the present invention is that the gelation is induced without denaturing the protein.

Another advantage of the present invention is that the gelation is induced without applying any heat treatment or enzymatic treatment to the protein therefore the method can be used in applications where heat sensitive proteins are used or bioactivity is sought to be conserved.

Another advantage of the present invention is that the gelation occurs at lower concentrations of proteins and polysaccharides than reported in the prior art for the gelation of protein-polysaccharide mixtures, or protein or polysaccharide solutions alone. In the industry, lower concentrations will allow to finely control the amount of active ingredients used and also to improve the efficiency of these ingredients. Moreover, using less of the ingredients is more economic.

Other objects and advantages of the present invention will be apparent upon reading the following non-restrictive detailed description, made with reference to the accompanying drawings.

### BRIEF DESCRIPTION OF THE FIGURES

FIG. 1 shows the time evolution of the storage modulus (G′) for 4-lactoglubulin (βIg)-xanthan gum mixtures at r=2 (◯), 5 (⋄), 15 (□) and 20 (+). FIG. 2 shows the time-evolution of the storage modulus G′ for βIg-xanthan gum mixtures at r=5, using native xanthan gum with M_{w}=5.1×10⁶ Da (⋄), or degraded xanthan samples with M_{w}=4.4×10⁶ Da (□) and Mw=3.2×10⁶ Da (Δ).

FIG. 3 shows the evolution of the storage [symbol] and loss (▪) modulus during gelation for βIg-xanthan gum mixtures at (a) r=2 and (b) r=5. The acidification curves (—) are also presented. The dotted lines indicate the gelation time (t_{gel}) defined as G′/G″ crossover. The IEP of βIg (pH=5.1) is also indicated (*).

FIG. 4 shows the phase contrast micrographs (40×) of the microstructure of βIg-xanthan gum gels for (a) r=2; (b) r=5 and (c) r=15. Bars are 40 μm.

FIG. 5 shows the evolution of the storage (G′) and loss (G″) modulus (0.1 Hz, 0.1% stress) during gelation time for β-lactoglobulin-xanthan gum mixtures at r=2:1 and 5:1.

FIG. 6 shows the gelation curves for BSA or βIg-λ-carrageenan systems at r=2 and 0.5 wt %.

FIG. 7 shows the Influence of the charge density of the utilized protein (a) βIg-Xanthan gel and (b) BSA-xanthan gel presenting syneresis.

FIG. 8 shows the phase contrast micrographs of (a) βIg-xanthan gel and (b) BSA-xanthan gel. The structure is clearly more compact with BSA. Bar 40 μm.

FIG. 9 shows the phase contrast micrographs of BSA-A-carrageenan gel (a) at 0.01M NaCl and (b) at 0.02M NaCl. The structure is clearly less compact at higher ionic strengths. Bar 40 μm.

FIG. 10 shows the gelation of different proteins with (a) xanthan gum and (b) gellan gum.

FIG. 11 shows the evolution of the storage modulus G′ (0.1 Hz, 0.1% stress) during gelation time for β-lactoglobulin-xanthan gum mixtures at r=5:1, using native xanthan gum —□— (Mw=5.1*10⁶ Da), or degraded xanthan samples [symbol] (Mw=4.4*10⁶) and [symbol] (Mw=3.2*10⁶ Da).

### DETAILED DESCRIPTION OF THE INVENTION

In describing the present invention, the following terminology will be used in accordance with the definitions set out below:

By “about”, it is meant that the value of said temperature, concentration or pH can vary within a certain range depending on the margin of error of the method used to evaluate such temperature, concentration or pH. For instance, the value for the temperature may have a variation of ±0.1° C. as read on a laboratory thermometer such as the one made by an ASTM™ thermometer. The value for the concentration may have a variation of ±0.01 wt % or ±0.001 M when the quantities of solutes and solvents are weighed on a laboratory scale such as the one made by Sartorius™ or when the quantities of solvents measured in a volumetric flask such as the one made for instance by Pyrex™.

r is the ratio of protein to polysaccharide.

Undenatured protein: relates in general to a native protein and more specifically to a protein that has not undergone a pre-treatment that modifies its structure such as a pre-heating treatment or enzymatic treatment.

Dispersion: is a mixture in which fine particles of one substance are evenly distributed throughout another substance such as, but not limited to water.

Gel: is a three-dimensional semi-solid structure formed by interconnected particles that restrict the movement of the dispersing medium.

Hydrogel: is a gel composed of either covalently or electrostatically cross-linked polymeric networks, which absorbs and retains large amounts of water.

Gelation: formation of a stable three-dimensional structure.

Gelation point: is the minimal total solids concentration (wt %) at which gelation takes place in a system with a fixed r.

pH_{gel}: pH at which a stable three-dimensional structure (i.e. a gel) is formed, under determined conditions of total solids and r.

pH_{φ}: pH at which intermolecular aggregation begins, i.e., when protein-polysaccharide soluble complexes aggregate into larger complexes.

G′: shear storage modulus, refers to the elastic character/stored energy of a material.

G″: shear loss modulus, refers to the viscous character/dissipated energy of a material.

G′/G″ crossover: Point at which G′ and G″0 have the same value. This condition usually indicates the formation of a three-dimensional network.

Rheology: is the study of the flow and deformation of matter, it describes the material properties of fluids and semi-solid materials.

Gradual modification: is a modification that is done by steps or degrees. For instance a gradual modification of the pH is modification by steps of the pH, contrary to the modification caused by a strong acid (e.g., HCl or H₂SO₄). A gradual modification of the electronic charge of a molecule such as a protein or a polysaccharide is a modification of one or more but not all charges in any one step of the modification.

Macromolecule: is usually an organic or inorganic molecule of high relative molecular mass, the structure of which comprises the multiple repetitions of units from molecules of low relative molecular mass. In the present invention it is preferably meant as an organic molecule and preferably an undenatured protein or a polysaccharide.

Protein isoelectric point (IEP): is the pH at which a molecule has no net charge and will not move in an electric field.

Refrigeration temperature: is the temperature at which usually development of microorganisms is hindered or stopped. It is usually meant to be about 4° C.

Room temperature: it is understood to be the normal ambient temperature of a laboratory or room where it would be comfortable for a human to work. It is meant to be about 23° C.

Electronic charge: it is meant the electric charge of a molecule and more specifically of the protein and the polysaccharide of the invention. For instance a protein and/or a polysaccharide may have a charge >0, =0 or <0.

Quiescent conditions: it is meant conditions where no disturbance occurs. In the present invention, a mixture or dispersion of the invention is left without being touched.

### Method of the invention

In a first embodiment, the present invention provides a method for gelation of an undenatured protein and a polysaccharide.

The method according to the invention first comprises the step of preparing a mixture of an undenatured protein and polysaccharide dispersions. The mixture is preferably obtained by mixing the protein dispersion with the polysaccharide dispersion according to a ratio of protein to polysaccharide preferably ranging from 1:1 to 50:1. The dispersions of the invention are prepared according to known methods in the art, such as by simply mixing a certain amount of the undenatured protein and the polysaccharide with water. The water used to prepare the dispersions of the invention is regular tap water, deionized, distilled or double distilled.

In a second step of the method of the invention, the mixture is stirred for a period of time to obtain a homogeneous mixture. It will be understood that stirring and/or mixing of the dispersions and mixture may be done by simply shaking the flask containing the dispersion or mixture, by using a common laboratory magnetic stirrer, by hand with a rod such as a glass rod, by using an automatic shaker or by any other laboratory mean suitable for stirring or mixing. By a “period of time”, it is understood any suitable period of time sufficient to allow the mixture to become homogeneous. As may be appreciated by a person skilled in the art, a mere addition of any one of the dispersions of the invention over the other may be enough to allow a sufficiently homogeneous mixture to form. As may also be appreciated, the period of time to allow the mixture to become homogeneous can for instance be as short as about 30 sec.

In a third step, the electronic charges of the undenatured protein and of the polysaccharide are gradually modified to obtain a mixture where the undenatured protein and the polysaccharide are oppositely charged.

In a fourth step, the homogeneous mixture obtained in the third step is let to rest without disturbance, i.e. under quiescent conditions, for a suitable period of time for gel formation. The gradual modification of the electronic charges of the undenatured protein and/or of the polysaccharide may also continue during this rest period. Gel formation will ensue after mixing has stopped.

As can be appreciated by a person skilled in the art, the period of time for gel formation depends on the nature of the undenatured protein and polysaccharide used. This period of time also depends on the concentration of undenatured protein and polysaccharide used and on the gradual modification of the electronic charge of the undenatured protein and/or the polysaccharide. As an example, the period of time suitable to form a gel in accordance to the present invention is in the range of about 5±1 min to about 1 hr±5 min. As can be appreciated also, some gels may need longer time to form.

According to the present invention, the undenatured protein dispersion is prepared without preheating the protein and/or without pre-treating it with an enzyme i.e. without denaturing it. When preparing the protein or the polysaccharide dispersions of the present invention, the protein and polysaccharide are dispersed in water as mentioned above. As may be appreciated by a person skilled in the art, the undenatured protein dispersion and the polysaccharide dispersion may be prepared separately or in the same dispersion. The dispersion(s) is(are) then allowed to hydrate for a suitable period of time. Such a suitable period of time allows hydration of the protein or the polysaccharide. As may be appreciated by a person skilled in the art, the time for hydration depends on the undenatured protein and polysaccharide used and on their concentration. For instance, the time used for hydration can be as short as about 0.5 hours or as long as about 30 hrs. In order to prevent molding or bacterial growth in the dispersions, the dispersions of the invention are preferably allowed to hydrate under refrigeration. As can also be appreciated by a person skilled in the art, the dispersions of the invention may alternatively be allowed to hydrate at room temperature, preferably in the presence of a bacteriostatic agent or any other agent known in the art to prevent bacterial growth or moulding in such dispersions. However, it will be understood that in the latter case if the gel obtained is to be used for applications in the food industry, the bacteriostatic used should be one approved for such use such as a benzoate, for instance sodium benzoate, sorbic acid or a sorbate, or a propionate for instance sodium propionate.

Gradually modifying the electronic charges of the undenatured protein and the polysaccharide according to the invention can take place at a temperature ranging from the refrigeration temperature to room temperature. As can be appreciated by a person skilled in the art, it is more comfortable to accomplish this step at room temperature.

According to another embodiment of the invention, stirring the mixture of the invention to obtain a homogeneous mixture, may also take place under refrigeration or at room temperature.

According to another embodiment of the invention, the homogeneous mixture obtained after stirring is let to rest under quiescent conditions for a period of time suitable to form a gel, under refrigeration or at room temperature. In a preferred embodiment, the homogeneous mixture obtained after stirring is let to rest at room temperature. As it may be appreciated, the modification of the electronic charges of the undenatured protein and/or polysaccharide may continue during this rest period. It is understood that for gels to be used in the food industry, the homogeneous mixture should preferably be let to rest under refrigeration. It is thus understood that gel formation according to the invention may take place under refrigeration or at room temperature.

According to the present invention the undenatured protein and polysaccharide dispersions have a relatively low protein or polysaccharide concentration (wt %). Preferably the concentration of the undenatured protein in the dispersion ranges from about 0.02 to 10 wt %. Preferably the concentration of the polysaccharide in the dispersion ranges from 0.02 to 5 wt %. For instance, the concentration of the undenatured protein or polysaccharide in each of the dispersions is about 0.1 wt %.

Also according to the present invention, the total concentration of undenatured protein and polysaccharide in the mixture is relatively low. Preferably the total concentration of the undenatured protein and polysaccharide in the mixture ranges from about 0.03 to 5 wt %. For instance, the total concentration of the undenatured protein and polysaccharide in a BSA-xanthan gum mixture is about 0.1 wt %.

According to the present invention, the gradual modification of the electronic charges is achieved by the gradual modification of the pH of the mixture. According to the invention, the pH of the mixture of the invention is adjusted to a level where it is favorable for attractive electrostatic interactions between different species (a pH close to or below the proteins' isoelectric point (IEP), when anionic polysaccharides are used, or to a pH close to or above the protein's IEP when cationic polysaccharides are used). At such conditions the system is said to be thermodynamically compatible. As may be appreciated by a person skilled in the art, gradual modification of the pH of the mixture of the invention means gradually lowering the pH or gradually increasing the pH. As may also be appreciated by a person skilled in the art, pH adjustment takes place and is carried out by a slow, gradual and preferably homogenous way to avoid the formation of large irregularities such as fibrous structures or large aggregates in the developing network.

According to the invention, gradual modification of the pH of the mixture of the invention may be achieved by the addition of a pH modifying agent. Such a pH modifying agent will allow a gradual and homogeneous pH modification and may thus be added to the mixture of the invention or to any of the dispersions of the invention. The amount of the pH modifying agent to be used according to the invention depends on the amount of undenatured protein and the amount of polysaccharide used. As may be appreciated by a person skilled in the art, the amount of the pH modifying agent may be increased if a faster modification of the pH and of the electronic charges of the undenatured protein and of the polysaccharide is sought. Also according to the invention, the gradual modification of the pH and hence of the electronic charges of the undenatured protein and/or polysaccharide continues through the period when the mixture is allowed to rest, for gel formation. Hence according to the present invention, the undenatured protein and the polysaccharide of the gel of the invention are oppositely charged.

According to the invention, for each protein to polysaccharide ratio tested, there is an optimum pH at which the firmness of the gel formed is maximal. For instance the pH of a mixture according to the invention can be from about 1.0 to about 5.5 for systems containing β-lactoblobulin (whose IEP occurs at pH=5.1).

In a preferred embodiment, the pH of a mixture or the dispersions according to the invention may be gradually lowered by the addition of a weak acid such as glucono-δ-lactone, some leavening agents or acid producing bacteria such as lactic acid producing bacteria, other acid producing bacteria such lactic acid producing bacteria for instance acetobacter, gluconobacter; or propionibacteria. In a more preferred embodiment of the invention, glucono-δ-lactone is used. Glucono-δ-lactone provides an acidification profile similar to that of lactic bacteria. Glucono-δ-lactone may be added to any of the dispersions or to the mixture according to the invention. As may be appreciated by a person skilled in the art, glucono-δ-lactone dissolves slowly and thus allows gradual and homogeneous lowering of the pH or acidification.

In a preferred embodiment, the glucono-δ-lactone is used at a concentration ranging from about 0.01 wt % to about 10 wt %. For instance, the concentration of glucono-δ-lactone used may be about 0.015 wt %. As may be appreciated by a person skilled in the art, the concentration of glucono-δ-lactone is preferably increased if faster acidification of the mixture or of any of the dispersions of the invention is sought. When the total protein and polysaccharide concentration is higher than about 0.1 wt %, the concentration of glucono-b-lactone used may be for instance higher than about 0.015 wt %.

In another preferred embodiment of the invention, and more specifically, in a case where the pH of the mixture of the invention has to be increased, a basic compound is used as a pH modifying agent. More specifically, a pH modifying agent such as sodium aluminum phosphate basic is used to increase the pH of a mixture that is prepared at a pH below the isoelectric point of the undenatured protein when a cationic polysaccharide is used. In a preferred embodiment, the polysaccharide is chitosan and the undenatured protein in βIg. More preferably, the pH of the mixture is close to 3.5 and is modified to about pH 6.

In accordance with the present invention the polysaccharide of the dispersion is preferably selected from the group consisting of polysaccharide of animal origin, polysaccharide of plant origin, polysaccharide of algal origin, polysaccharide of bacterial origin; and any mixture thereof. More preferably, the polysaccharide is selected from the group consisting of xanthan gum, gellan gum, λ-carrageenan and κ-carrageenan, I-carrageenan, alginates, pectins, carboxymethylcellulose, agar-agar, arabic gum, hyaluronates and any mixture thereof.

In accordance with the present invention, the undenatured protein is preferably any charged protein and more preferably selected from the group consisting of milk protein, plant protein and animal protein. According to a more preferred embodiment of the invention the undenatured protein is preferably selected from the group consisting of BSA, ovalbumin, β-lactoglobulin, soy protein, sodium caseinate, calcium caseinate, whey protein isolate, whey protein concentrate and gelatin.

According to a preferred embodiment of the invention, the ionic strength of the mixture can be increased. In some cases, an increased ionic strength allows the formation of more stable gels (i.e. without syneresis). In a preferred embodiment, the ionic strength is increased by adding a salt selected from the group consisting NaCl, KCl, CaCl₂, NH₄Cl, MgCl₂ and NaNO₃. In a more preferred embodiment NaCl is used, preferably at a concentration higher than about OM but lower than about 0.5 M

In another embodiment, the present invention relates to a gel obtained by the method of the invention. The gel of the invention is preferably an hydrogel since, as defined above, may be composed of either covalently or electrostatically cross-linked polymeric networks, which absorb and retain large amounts of water. In a preferred embodiment of the invention the concentration of protein and polysaccharide in the gel varies from about 0.03 to about 10 wt % and more preferably the concentration is <3.0 wt %. As one skilled in the art may appreciate, such a concentration is advantageously much lower than what is taught in the prior art.

As one skilled in the art may appreciate, the gel or the hydrogel of the invention finds advantageous applications as a matrix in the pharmaceutical industries for instance for the production of carrier matrices (caplets, patches, etc.) to deliver and protect drugs or active molecules (enzymes, antibodies, peptides etc.) and/or to enhance the stability of foods in the food industries, for the entrapment and/or protection of micronutrients (such as minerals, vitamins, peptides etc.) and in the production of cosmetics. Such a gel finds also an application in the formation of a film for product protection such as product protection against dehydration.

The following examples serve to illustrate the extent of the use of the present invention and not to limit its scope. Modifications and variations may be made without forgetting the intent and the extent of the invention. Even though other methods or equivalent products equivalent to those that are found herein to test or to realize the present invention may be used, the materials and the preferred methods are described.

### EXAMPLE 1

### Gelation of β-lactoglobulin with Xanthan Gum

### Materials

A high βIg content whey protein isolate was used as the source of βIg (High-Beta, lot # JE 002-8-922, 98.2 wt % protein, of which 85% is βIig, 1.8% minerals and 4% moisture, Davisco Foods International, Inc., MN, US). Due to the high content of βIg in this powder, it was assumed that its behavior was governed by that of βIg and therefore it will be referred to as βIg hereafter. Xanthan gum (Keltrol F, lot #9D2192K, 96.36% total sugar, 3.02% protein) from KELCO Co. San Diego, Calif.

Dispersions of βIg and xanthan gum containing 0.1 wt % total biopolymer concentration were prepared in filtered deionized water (Milli-Q, Millipore, US), left overnight at 4° C., then centrifuged and filtered, as previously described (Laneuville et al. (2005)), before preparing the mixtures for analysis. Different protein to polysaccharide ratios (r) were studied, namely 2:1, 5:1, 15:1 and 20:1 (r=2, 5, 15 and 20 respectively). βIg and xanthan gum dispersions (0.1 wt %) were stirred at the desired r and mixed gently for 30 min. Tests with microfluidized xanthan gum, prepared and characterized as previously described (Laneuville et al. (2000)), were also carried out for r=5. The initial pH of the mixed dispersions was 6.60±0.08. Electrostatic interaction was induced by slow acidification using 0.015 wt % glucono-δ-lactone acid (GDL) (Merck, Darmstadt, Germany) to a final pH=4.5. After the addition of GDL, dispersions were slowly stirred for an additional 15 min before starting rheometry or turbidity measurements. The pH of the dispersions was also followed in order to determine the acidification rate for each system. βIg and xanthan gum dispersions were tested separately for comparison.

### Dynamic Oscillatory Measurements

Small-deformation oscillatory measurements were performed in parallel to light scattering experiments using a stress controlled rheometer (Stresstech, Rheologica Instruments, Inc. Lund, Sweden) using a parallel plate geometry (UP30stried fixture, 20 mm diameter). βIg-xanthan gum mixtures with GDL were poured onto the bottom plate, the gap used was 1 mm. The temperature of the bottom plate was controlled with a Peltier system and maintained at 20° C. Samples were covered with a protective jacket to reduce evaporation during measurement. Oscillation experiments were conducted at a frequency of 0.1 Hz and a constant strain of 0.1%. At the end of oscillation, a strain sweep test was recorded to verify the linear region, which was taken as the stress at which the storage modulus was independent of strain. The formation of the gel network was followed by the development of G′ and G″ with time. Since some of the samples presented very tenuous networks, a strain at the beginning of the linear region was chosen to avoid rupture of the gel.

Overall, results were analyzed in terms of the temporal evolution of viscoelasticity and final structure as observed by microscopy.

### Phase Contrast Optical Microscopy

Phase contrast optical micrographs were taken using a BX-51 optical microscope (Olympus, Tokyo, Japan) at a 40× magnification. GDL was added to the βIg-xanthan gum mixtures and mixed for 15 minutes. Then samples were placed onto microscope slides, covered, and sealed with nail enamel. Micrographs were taken ˜18 h after GDL addition. At that time, the structure of the gels was fully developed.

### Results and Discussions

Time Evolution of Viscoelasticity and Critical pH_{φ}

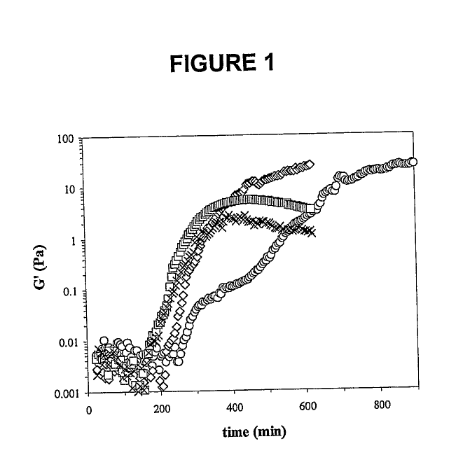

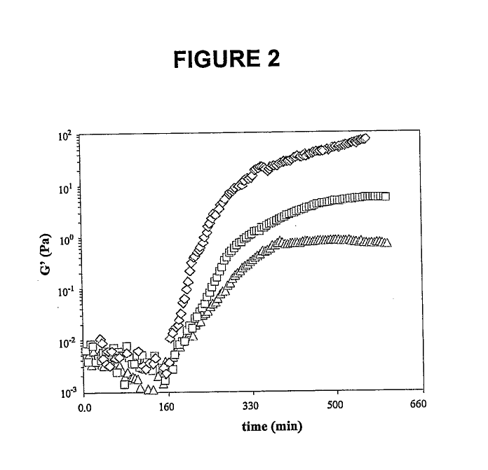

Storage (G′) and loss (G″) modulus development was followed over the course of acidification at different protein to polysaccharide ratios (r). FIG. 1 presents the G′ time-evolution for all the tested r. In general, increasing protein content resulted in softer and more opaque gels, possibly due to the disruption of the network by excess protein or βIg-xanthan complexes. Stable gels were obtained for r=2 and r=5. The gels with the highest G′ were obtained at r=2, these gels were transparent, whereas at r=5, the gels were semi translucent. The gels obtained at r=15 presented a lower solid-like character and were opaque. Gels formed at r=15 broke up into flocs when vigorously shaken in a test tube, the flocs soon transformed into particulated complexes that precipitated. At sufficiently high r (r=20), gelation did not occur; instead, precipitated electrostatic complexes formed from the beginning. The increase of G′ detected at r=20 might be related to a structuration of the system.

FIG. 2 presents the evolution of G′ during gel formation for systems containing microfluidized xanthan gum. It can be seen that for the same protein to polysaccharide ratio r, softer gels (lower G′) were formed when microfluidized xanthan was used. Moreover, it can clearly be seen that for r=5 with native xanthan, there is an important structuration process occurring at t˜180 min, characterized by a steep increase in G′.

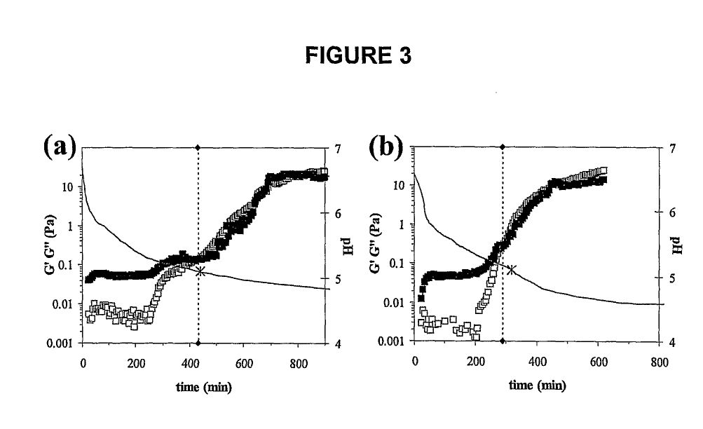

FIGS. 3a and 3b show the G′ and G″ evolution for samples at r=2 and r=5. The acidification curves and the time at which the IEP of βIg is attained (pH 5.1) are also presented. Table 1 presents the gelation time (t_{gel}) defined as the G′/G″ crossover, its corresponding pH_{gel}, the critical pH_{φ} determined from turbidity, and other measured physical parameters for all the studied r.

TABLE 1
Time (t_{gel}) and pH (pH_{gel}) of gelation; critical pH_{φ};

| Ratio | t_{gel} (min) | pH_{gel} | pH_{φ}^{*} |
|---|---|---|---|
| 2 | 442.0 ± 8 | 5.07 ± 0.01 | 5.16 ± 0.01 |
| 5 | 263.0 ± 12 | 5.21 ± 0.01 | 5.30 ± 0.04 |
| 15 | 284.8 ± 21 | 5.24 ± 0.06 | 5.49 ± 0.01 |
| 20 | — | — | 5.49 ± 0.04 |

*Obtained from turbidimetry studies

From FIGS. 3a and 3b, it can be seen that the G′/G″ crossover always occurs around the IEP of βIg, with a tendency to occur slightly above the proteins' IEP, at pH˜5.24, for high protein content systems (Table 1). However, it is evident that the structuration process begins well above the IEP. It was found that G′ initiated its increase at around pH_{φ}, the latter being determined from turbidity measurements (Table 1) and occurred above the proteins' IEP for all the studied ratios. This is in agreement with previous results, that showed that the pig possesses charged patches (Girard et al. (2003)) susceptible to interact with xanthan gum above its IEP (Laneuville et at. (2005)), and is also in agreement with several results on other protein-polysaccharide systems (Weinbreck et al. (2003a); Girard et al. (2003); Mattison et al. (1999)).

Although pHc could not be determined; it was obvious that turbidity was increasing before pH_{φ}, indicating the formation of soluble complexes. Additionally, the measured pH_{φ} (Table 1) are in remarkable good agreement with those found for the same systems under shear (Laneuville et al. (2005)), showing that, at least in the beginning, the electrostatic interaction between βIg and xanthan gum follows the same path either under shear or under quiescent conditions, i.e., the formation of soluble complexes followed by interpolymeric complexation. The gelation point (G′/G″ cross-over) seems to take place after both these processes have occurred. However, for low r, the gelation kinetics is faster than the separation kinetics, thus the structure is trapped before it can completely phase separate into electrostatic complexes as occurs for high r. Also noticeable is that there is a pH at which G′ and G″ seem to attain an equilibrium (FIG. 3). For r=2 and r=5, this pH is ˜4.8±0.08 and indicates that the gel structure is stabilized when both molecules carry net opposite charges.

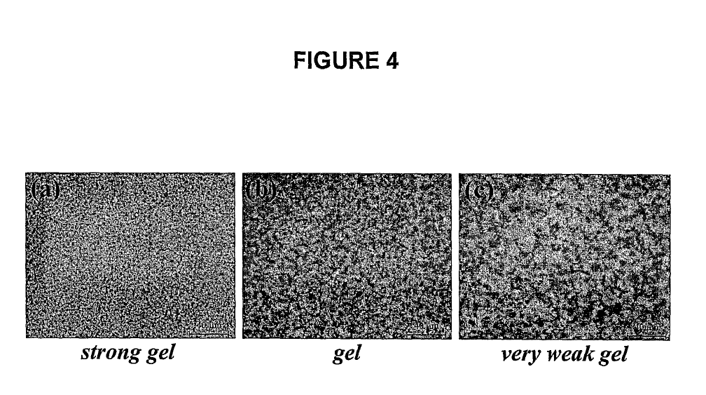

The pH at which a maximum in G′ is obtained, corresponds to the stoichiometric electrical charge equivalence pH (EEP), where molecules carry similar but opposite charges and the interaction is at its maximum (Mattison et al. (1999); Burgess (1994)). At the EEP, there is an electrostatic equilibrium in the gel, due to the balance of attractive and repulsive forces that results in a stable gel, composed mainly of aggregated complexes of fractal nature. At higher r, the excess protein affects this equilibrium, and hinders gel stability by favoring strong interactions between protein and polysaccharide molecules, thereby leaning the equilibrium towards the formation of particulated complexes (Laneuville et al. (2000)). This explanation is supported by the lower G′ values and the higher opacity obtained for gels with high protein content. Owing to the fact that the EEP is controlled by the number of opposite charges in the system; it is not surprising to find that there is an optimal r at which stability is maximal. Accordingly, gels formed at r=2 and r=5 were stable over a large range of pH, as seen in FIGS. 3 and 4, and presented higher G′ values (FIG. 1).

FIG. 4 presents phase contrast micrographs for r=2, 5 and 15. It can be seen that the internal structure of the gels contain larger and denser structures as the content of protein increases. At r=2, the microstructure presents very diffuse interfaces, the high xanthan content in this sample may have played a role in reducing the mobility of the interacting clusters, resulting in a finer and more homogeneous network. At higher r, the interfaces are better defined and large electrostatic complexes can be observed. The formation of larger electrostatic complexes at high r (Laneuville et al. (2000) and (2005)) seems to disrupt the gel network, resulting in the lower G′ measured by rheometry (FIG. 1) and eventually completely hindering gel formation as found for r=20.

The junction zones among clusters are formed due to opposite charges of βIg and xanthan gum and also by the bridging of clusters that share different portions of the same xanthan molecules. Hydrogen bonding and other non-covalent interactions may further stabilize the gel.

The gelation process would be a competition between the phase separation process, which is set off by the increasing electrostatic interaction between protein and polysaccharide molecules as pH decreases, and the gelation that arrests coarsening and phase separation. This scenario has some similarities to that encountered in systems where the forces leading to phase separation are segregative (Anderson and Jones, (2001); Hong and Chou (2000); Kita et al. (1999); Asnaghi et al. (1995)).

### Conclusions

The kinetics of the cold gelation of β-lactoglobulin and xanthan aqueous mixtures are studied by rheometry. The interaction between βIg and xanthan under quiescent conditions started at positively charged patches on the protein surface, before the isoelectric pH of βIg. Initially, primary complexes, with a diffuse structure, formed, then, they aggregated into more dense interpolymeric complexes that formed caged clusters with low mobility, the clusters formed junction zones and the whole structure was freeze-in at the point of gelation. The βIg-xanthan ratio had an important effect on the reaction rate and the stability of the gels. An optimal ratio was found for which the gels were stable over a large range of pH. This was related to the existence of a stoichiometric electrical charge equivalence pH.

### EXAMPLE 2

In this example, studies were made on systems of different proteins mixed with three different polysaccharides, namely xanthan gum, gellan gum and λ-carrageenan. In this example, studies were made on different systems to determine the extent of the gelation zone. Several protein to polysaccharide ratios (r=1:1 to 50:1) were studied at different total solids concentrations (0.05 to 3 wt %) and different ionic strengths (0.0-0.5 M NaCl). The point of gel, i.e., the minimal concentration at which gelation can occur, for all studied systems was determined at different protein to polysaccharide r, namely 1:1, 2:1 and 3:1. Native xanthan gum (Mw=5.1*10⁶ Da) as well as degraded xanthan samples with lower molecular weights (Mw=4.5; 3.9 and 3.2*10⁶ Da) were also tested. The experiments were carried out at constant temperature and two temperatures were tested: room temperature (˜23° C.) or refrigeration temperature (4° C.). Gelation was induced by in-situ pH adjustment to a pH where both molecules carry net opposite charges. The formation of a gel was followed by dynamic rheology and microscopy. The firmness, transparency/opacity and internal structure of the gels is tailored by adjusting the pH, the protein to polysaccharide ratio (r), ionic strength, total solids concentration, etc. However, they depended principally on the charge density of the reacting molecules and therefore, were most sensitive to pH and ionic strength.

### Materials and Methods

The mixing procedure for the systems studied in this example varied slightly than in Example 1, but is essentially equivalent. In this case glucono-δ-lactone (GDL) was dissolved in the protein dispersion prior to mixture, this allowed an easier GDL dissolution, especially when higher concentrations (0.2-0.5 wt %) were tested.

Dispersion of protein and polysaccharide at the required concentration (0.2-0.5 wt %, depending on the system) were prepared in filtered deionized water (Milli-Q, Millipore, US) and left overnight to allow complete hydration, at 4° C., to prevent mould or bacterial growth. The adequate quantities of the polysaccharide and protein dispersion to obtain r=2 were measured in separate beakers, the GDL was added to the protein dispersion alone and mixed for 30 sec. then the protein+GDL was poured onto the polysaccharide dispersion under continual stirring, and stirring continued for another 1.5 minutes. Then samples were placed on the rheometer geometry or on the microscope slides.

Phase contrast optical micrographs were taken using a BX-51 optical microscope (Olympus, Tokyo, Japan) at a 40× magnification. Samples were placed onto microscope slides, covered, and sealed with nail enamel. Micrographs were taken ˜20 h after GDL addition. At that time, the structure of the gels was fully developed.

Dynamic Oscillatory measurements were performed at 23° C. with a shear-rate controlled rheometer (ARES-100FRT, Rheometric Scientific, Piscataway, N.J.) equipped with a couette-type sensor. The inner and outer cylinder radiuses were 33 and 34 mm respectively; the length of the inner cylinder was 33 mm. Protein-polysaccharide mixtures with GDL were poured onto the bottom cylinder, the gap used was ˜7 mm. Samples were covered with a protective jacket to reduce evaporation during measurement. Oscillation experiments were conducted at a frequency of 1 Hz and a constant strain of 0.5%. At the end of oscillation, a frequency sweep test (0.01-15 Hz at 0.5% strain) and a strain sweep test (0.01 to 100% strain at 1 Hz) were recorded to verify the linear region, which was taken as the stress at which the storage modulus was independent of strain. The formation of the gel network was followed by the development of G′ and G″ with time. The G′/G″ crossover was taken as the gel point.

The utilized materials for this Example were:
Calcium-Caseinate (Ca-caseinate): ALANATE 380, from Nealanders International Inc. Montreal Canada, lot 4174-X3108. 96.7% protein dry basis.
Sodium-Caseinate (Na-caseinate): ALANATE 180, from Nealanders International Inc. Montreal Canada, lot 4674X1006. 96.9% protein dry basis.
Whey protein concentrate (WPC): from Davisco Foods International Inc., Le Sueur, Minn., US, lot 009-5-280, 81.2% protein dry basis.
Gelatin (Type B, from bovine skin): from Sigma-Aldrich, lot 129H1404.
Albumin from bovine serum (BSA): from Sigma-Aldrich, lot 074K0567, min 98% protein.
Ovalbumine (Albumin from chicken egg white, Grade V) from Sigma-Aldrich, lot 122K7044, min 98% protein.
β-lactoglobulin (βIg) High—Beta, lot #JE 002-8-922, 98.2 wt % protein dry basis, from Davisco Foods International, Inc., MN, US.
λ-carrageenan: (Irish moss, type IV) from Sigma-Aldrich, lot 122K1444.
Gellan gum: Kelgogel F (low-acyl gellan gum) from CP Kelco, San Diego, Calif.
Xanthan R D: Keltrol R D, from CP Kelco, San Diego, Calif.

### Results

### Effect of the Protein-to-Polysaccharide Ratio

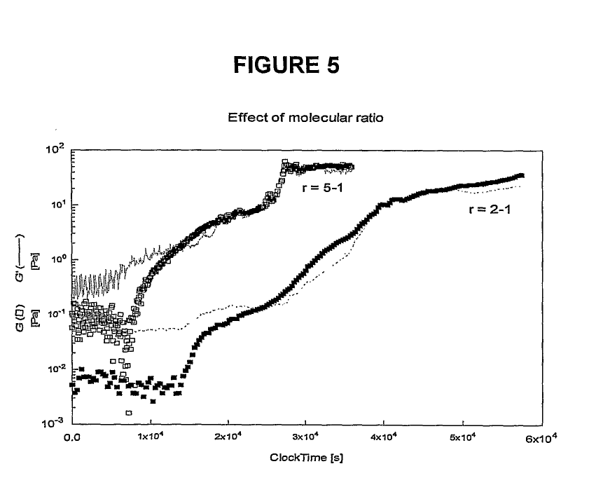

There is an optimal protein to polysaccharide ratio for which the viscoelasticity of the gel is maximal (FIG. 5). It is assumed that the firmness is at its maximum at a stoichiometric ratio, which generally approaches a 1:1 ratio. Higher ratios resulted in softer and more opaque gels, due to the disruption of the network by excess protein. Whereas at lower ratios, gel formation capability was rapidly reduced and eventually the mixture presented a viscoelastic profile that resembled that of the polysaccharide dispersion alone, i.e., a polysaccharide dispersion whose rheological properties are essentially governed by molecular entanglements. This effect is also evident in Table 2, where it can be seen that the optimum protein to polysaccharide ratio (r) i.e, the r at which the lowest concentration permitting the formation of a gel is found, is often r≈2 for systems containing xanthan and gellan gum, whereas for systems with λ-carrageenan the optimal r≈3. The upper protein to polysaccharide ratio limit for gel formation varies according to the protein-polysaccharide system, concentration and ionic strength. In general, protein to polysaccharide ratios<50:1 should be used, with an optimal ratio within 1:1 and 20:1.

### Effect of the Ionic Strength

Ionic strength has a great influence on gelation due to the ionic nature of the protein-polysaccharide interactions involved in the stabilization of the gelified structures. At higher ionic strengths, the optimum r is shifted to r=1 (Table 2). This is due to the shielding of reactive groups on the molecules, and therefore more protein is required to achieve a better level of interaction and gel firmness. In increasing the ionic strength, gels were less firm and more opaque since at higher ionic strengths, less reactive sites are available for interaction, due to charge shielding, and less junction zones could form to stabilize the network. The opacity of the gels increased due to the formation of larger structures. Therefore there is a limiting ionic strength above which gelation will not occur due to counterion charge screening. The upper limit of ionic strength varies according to the protein-polysaccharide system, concentration and protein to polysaccharide ratio. For example, in the case of BSA-xanthan gum systems, higher ionic strengths could be used, and still obtain a firm gel, due to the high charge density of this protein compared to the other tested proteins. At a concentration of 0.2 wt % and r=1, ionic strengths as high as ˜0.20M is used with BSA-xanthan systems, whereas with βIg-xanthan systems, the maximal ionic strength that allowed gel formation, was ˜0.08M NaCl. In general, ionic strengths <0.5M should be used, with an optimal ionic strength that varies depending on the concentration and the utilized system. Higher concentrations allow the use of higher ionic strengths.

### Effect of Total Solids Concentration

Gelified systems could be obtained from very dilute mixtures, ˜0.03 wt % total solids (Table 2). Viscoelasticity and opacity of the gels increased with increasing total solids content due to an increase in junction zones. At 0.1 wt % total solids, some gels presented a remarkable high viscoelasticity and stability. These gels are also hydrogels due to their high water content. Very firm gels are obtained by increasing the total solids concentration, and higher r could be used. For example, at 0.1 wt % the maximum r at which a gel can be formed in a βIg-xanthan gum mixture is r=15, whereas at 1% the maximum r at which a gel can be formed for this system is r˜50.

### Effect of the Molecular Weight of the Polysaccharide

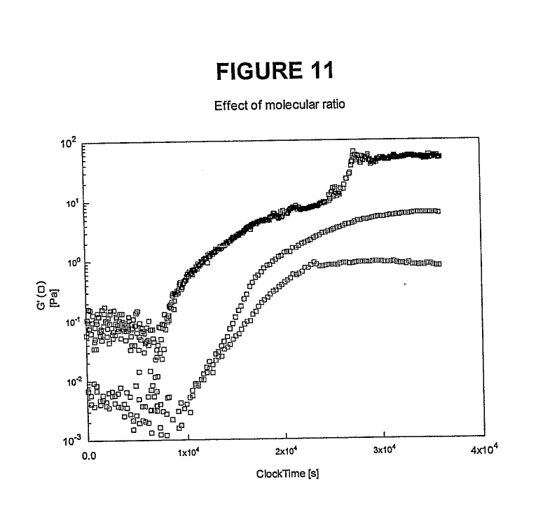

Varying the molecular weight and aggregated state of the polysaccharide had also an effect on the characteristics of the gels. For the same protein to polysaccharide ratio and total solids content, softer gels resulted when lower molecular weight polysaccharides were used (FIG. 11). There is a limiting Mw required for the formation of a gel below which particulated electrostatic complexes will form instead of a stable network.

### Effect of the Temperature

No effect of the temperature (4° C. vs 23° C.) was noticed on the gel formation.

### EXAMPLE 3

In this example, studies were made on systems of different proteins mixed with three different polysaccharides, namely xanthan gum, gellan gum and λ-carrageenan. Specifically, dynamic rheology allowed to detect the effect of the different proteins and polysaccharides structures. The protein to polysaccharide r studied was set to r=2, the total solids concentrations tested were of 0.2, 0.3 and 0.5 wt % for systems containing xanthan, gellan and λ-carrageenan respectively. The gelation process was followed by dynamic rheology and microscopy. The viscoelasticity, transparency/opacity and internal structure of the gels were tailored by adjusting the initial protein and polysaccharide. However, they depended principally on the charge density of the reacting molecules and therefore, were sensitive to pH and ionic strength. Gelation was induced as in example 2. The materials and methods utilized are the same as those presented in the example 2.

### Results

### Effect of the Protein Charge Density

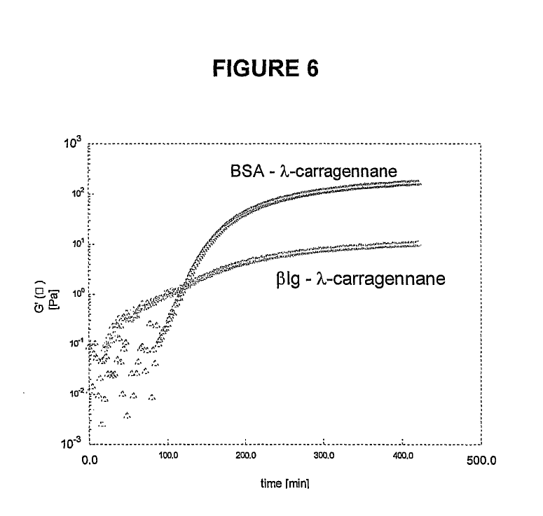

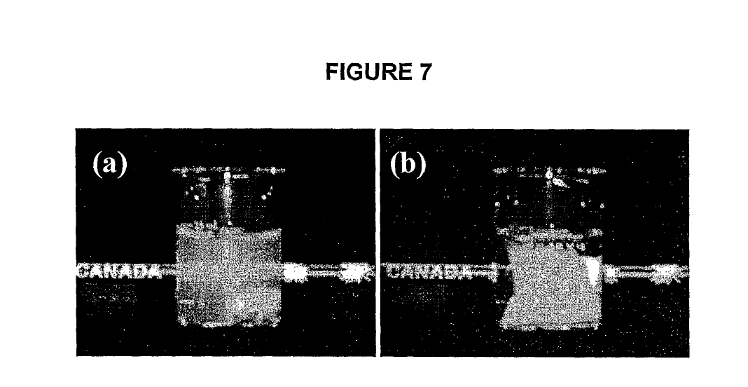

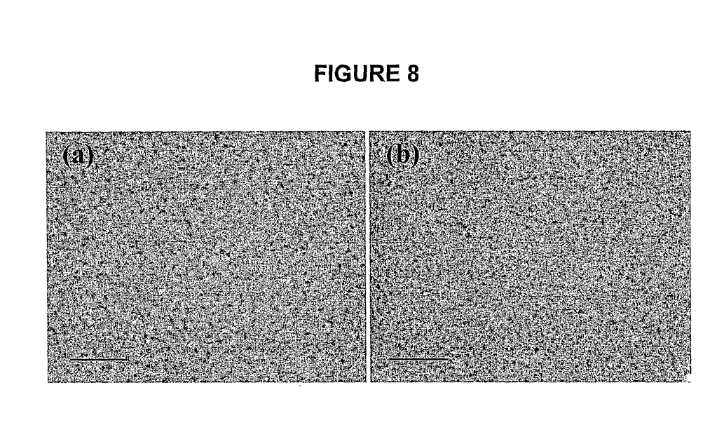

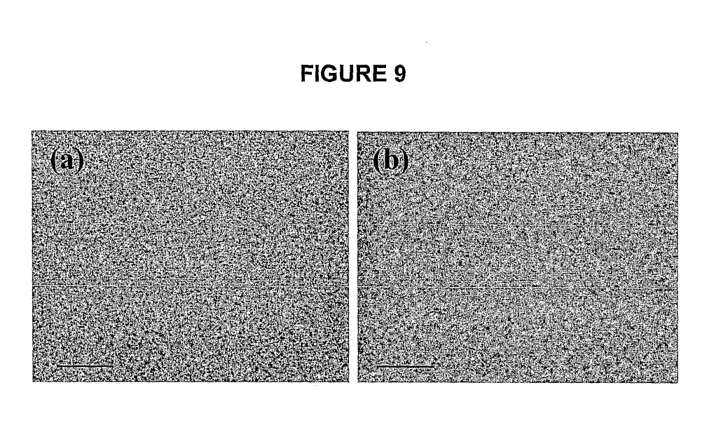

The protein charge density appears to have an influence in the firmness and stability of the gels. Specifically, when comparing the gel formation between βIg and BSA (both globular proteins) with λ-carrageenan, firmer gels are obtained with BSA (FIG. 6). This may possibly be due to the higher charge density of BSA compared to βIg, in part also since BSA is a larger molecule and therefore has more reactive sites. This effect is also evident in systems with xanthan and gellan gum, however the most important effect is observed with λ-carrageenan. Furthermore, when a protein with several reactive sites, such as BSA (compared to βIg), is used to form this type of gel, the interaction is so strong, that often the gel shrinks and expels water (FIG. 7). Microscopy studies show that the gel structure formed in BSA-xanthan systems is indeed much more compact than the one formed in βIg-xanthan systems (FIG. 8). In order to overcome syneresis, it is possible to increase the ionic strengths, e.g., to 0.02M NaCl, which results in the formation of homogeneous and stable gels with BSA due to the shielding of some of the charges on the molecules, therefore reducing the strength of the interaction (FIG. 9). Proteins with higher charge densities result in tighter structures, due to increased junction zones. Charge density is influenced by pH, therefore, the optimal pH of interaction (at which the firmest gel can be formed) varies from protein to protein.

### Effect of the Protein Conformation

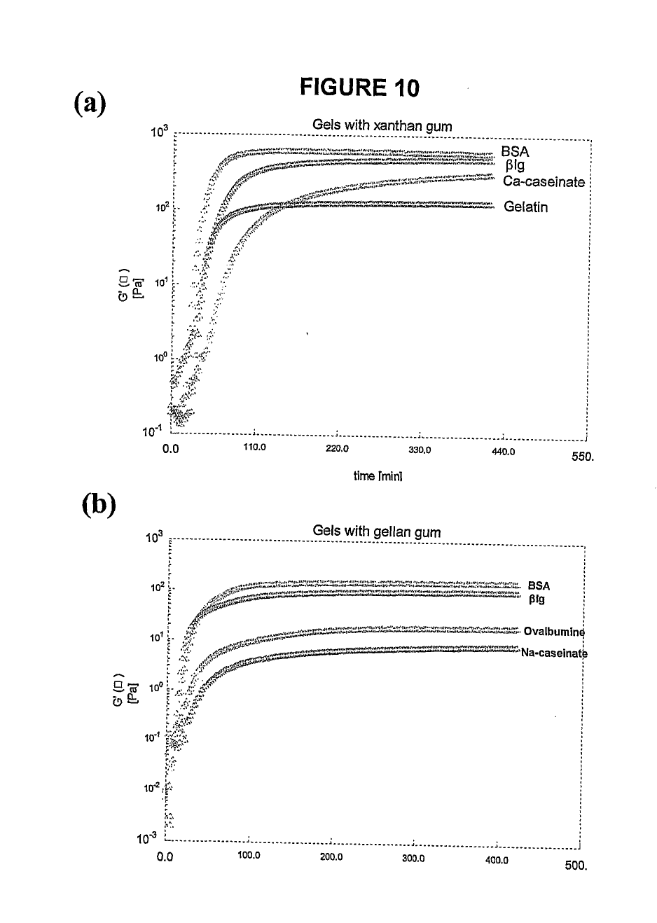

The conformation of the protein has an impact on the firmness of the gels, in general globular proteins (BSA, βIg, Ovalbumin) form stronger gels than linear proteins (sodium or calcium caseinate, gelatin) (FIG. 10). However, this effect is coupled with the charge density of the protein.

### Effect of the Polysaccharide Conformation

The conformation of the polysaccharide appears to have a great importance in the gelation ability of the system. When long, stiff polysaccharides are used, e.g., xanthan gum, a gel can be formed; otherwise particulated complexes or coacervates will form, e.g. when acacia gum (a more flexible polysaccharide) is used. The ability to form tenuous networks in dispersion is important, since this is responsible for stabilizing the gel structure by preventing an over-aggregation, which would lead to gel weakening. In general, xanthan and gellan gum seem to be more suitable for gel formation than λ-carrageenan, the latter being able to form gels at higher concentrations (Table 2). This would also explain why the effect of using BSA is more pronounced when λ-carrageenan is used. Xanthan and gellan gum are very stiff polysaccharides know to aggregate in solution, whereas λ-carrageenan is more flexible, although it can form double helical aggregates at higher concentrations. It is however important to note that when globular and highly charged proteins (i.e., BSA) are used in conjunction with λ-carrageenan, gels can be obtained at fairly low concentrations (<0.3 wt %) (as seen from Table 2).

### EXAMPLE 4

Using a mixture of chitosan (a cationic polysaccharide) and βIg at 0.2 wt %, the mixture is prepared at a pH below the isoelectric point of the protein, e.g., at pH=3.5, then an alkalizing agent is added, the system is mixed to obtain an homogenous mixture and then it is let at rest to allow gel formation. The final pH of the systems should be around pH 6. The alkalizing agent is for example sodium aluminium phosphate basic

TABLE 2
Gel point (minimum total wt % at which gelation occurs) for several protein-
polysaccharide systems. The lowest obtained concentrations for gel formation
with each polysaccharide is indicated by bold lettering.

| | | Xanthan gum | | Gellan gum | | λ-carrageenan | |
|---|---|---|---|---|---|---|---|
| | | | Gel point | | Gel point | | Gel point |
| | | | (0.02M | Gel | (0.02M | | (0.02M |
| Protein | ratio | Gel point | NaCl) | point | NaCl) | Gel point | NaCl) |
| Na-Caseinate | 1 | 0.05 | 0.10* | 0.55 | 0.12 | 1.30 | n.d. |
| | 2 | 0.05 | 0.10* | 0.65 | 0.15 | 1.10 | |
| | 3 | 0.08 | 0.10* | 0.60 | 0.18 | 1.10 | |
| Ca-Caseinate | 1 | 0.06 | 0.07 | 0.28 | 0.10 | 1.30 | n.d. |
| | 2 | 0.05 | 0.08 | 0.25 | 0.12 | 0.90 | |
| | 3 | 0.06 | 0.09 | 0.18 | 0.12 | 0.80 | |
| BSA | 1 | 0.04^{§} | 0.045 | 0.09 | 0.035 | 0.50 | n.d. |
| | 2 | 0.03^{§} | 0.045 | 0.085 | 0.04 | 0.40 | |
| | 3 | 0.03^{§} | 0.05 | 0.09 | 0.05 | 0.40^{§} | |
| βlg | 1 | 0.05 | 0.10 | 0.10 | 0.06 | 0.60 | n.d. |
| | 2 | 0.035 | 0.15 | 0.09 | 0.06 | 0.40 | |
| | 3 | 0.04 | 0.20 | 0.12 | 0.07 | 0.40 | |
| WPI (80% P) | 1 | 0.05 | 0.09 | 0.07 | 0.04 | 1.20 | n.d. |
| | 2 | 0.04 | 0.10 | 0.065 | 0.05 | 0.90 | |
| | 3 | 0.05 | 0.14 | 0.07 | 0.07 | 0.80 | |

*In these systems gels with important syneresis were obtained at 0.06-0.07 wt %, stable gels formed at 0.1 wt %.
^{§}These systems presented important syneresis which may be corrected by increasing the ionic strength.
n.d. = not determined, the wt % concentration required for theses systems to form a gel are high (≧1.2 wt %) and therefore are not as interesting.
The lowest obtained concentrations for gel formation in each system is indicated by bold lettering.

### REFERENCES

Alting A. C. De Jongh H. H. J., Visschers R. W., Simons J. W. F. A. (2004). Acid-induced cold gelation of globular proteins: effects of protein aggregate characteristics and disulfide bonding on rheological properties. Journal of Agricultural and Food Chemistry, 52: 623-631.

Alting A. C. De Jongh H. H. J., Visschers R. W., Simons J. W. F. A. (2002). Physical and chemical interactions in cold gelation of food proteins. Journal of Agricultural and Food Chemistry, 50, 4682-4689.

Anderson V. J., and Jones R. A. L. (2001). The influence of gelation on the mechanism of phase separation of a biopolymer mixture. Polymer, 42, 9601-9610.

Asnaghi D., Giglio M., Bossi A. and Righetti P. G. (1995). Large-scale microsegregation in polyacrylamide gels (spinodal gels). Journal of Chemical Physics, 102 (24): 9736-9742

Baeza, R.; Gugliotta L. M.; Pilosof, A. M. R. (2003). Gelation of β-lactoglobulin in the presence of propylene glycol alginate: Kinetics and gel properties. Colloids and Surfaces B: Biointerfaces, vol. 31 (1-4), 81-93.

Berger J., Reist M., Mayer J. M., Felt O., Gurny R. (2004) Structure and interactions in chitosan hydrogels formed by complexation or aggregation for biomedical applications. European Journal of Pharmaceutics and Biopharmaceutics, 57, 35-52.

Britten M. and Giroux H. J. (2001). Acid-induced gelation of whey protein polymers: effects of pH and calcium concentration during polymerization, Food Hydrocolloids, 15 (4-6), 609-617.

Bryant C. M. and McClements D. J. (2000) Influence of xanthan gum on physical characteristics of heat-denatured whey protein solutions and gels. Food Hydrocolloids, Vol. 14, (4), 383-390.

Burgess D. J. (1994). Complex Coacervation: Micro-Capsule Formation. In: Macromolecular Complexes in Chemistry and Biology; Chap. 17, pp. 285-300. Dubin P., Bock J., Davies R. M., Schulz D. N. & Thies C. (Eds.), Springer, Berlin.

Chen J., Jo S., and Park K. (1995). Polysaccharide hydrogels for protein drug delivery. Carbohydrate Polymers, 28, 69-76.

Desrosiers E. A., Chenite A., Berrada M., Chaput C. US 2004/0091540 A1. Published on May 13, 2004. Method for restoring a damaged or degenerated intervertebral disk.

Dickinson E. (1998). Stability and rheological implications of electrostatic milk protein-polysaccharide interactions. Trends in Food Science and Technology, Vol. 9, Iss. 10, 347-354

Dickinson E. (2003). Interfacial, emulsifying and foaming properties of milk proteins. In: Advanced Dairy Chemistry: Proteins, Vol. 1, Part B, Chap. 27, pp. 1229-1260. 3^{rd} edition. Fox P. F. and McSweeney P. L. H. (Eds.), Kluwer Academic/Plenum Publishers, New York. 603p.

Dickinson E. and McClements D. J. (1996). Protein-Polysaccharide interactions. In: Advances in Food Colloids. Blackie Academic and Professional. Chapman and Hall, Glasgow. 350 pp.

Dumitriu S. and Chornet E. (1998) Inclusion and release of proteins from polysaccharide-based polyion complexes. Advanced Drug Delivery Reviews, 31 223-246.

Edman P., Ekman B. and Sjoholm I. (1980). Immobilization of proteins in microspheres of biodegradable polyacryidextran. J. Pharm. Sci. 69, 838-842.

Eissa A. S., Bisram S., Khan S. A. (2004) Polymerization and gelation of whey protein isolate at low pH using transglutaminase enzyme. Journal of Agricultural and Food Chemistry 52, 4456-4464.

Foegeding E. A., Davis J. P., Doucet D. and McGuffey M. K. (2002) Advances in modifying and understanding whey protein functionality. Trends in Food Sci. and Tech., Vol. 13 (5), 151-159.

Girard M., Turgeon S. L., and Paquin P. (2002). Emulsifying properties of Whey Protein-Carboxymethylcellulose complexes. J. of Food Science. Vol. 67, No. 1, pp. 113-119.

Girard M., Turgeon S. L. & Gauthier S. F. (2003). Quantification of the Interactions between β-Lactoglobulin and Pectin through Capillary Electrophoresis Analysis. Journal of Agricultural and Food Chemistry, 51: 6043-6049.

Gombotz W. R., and Wee S. F. (1998) Protein release from alginate matrices. Advanced Drug Delivery Reviews 31, 267-285.

Hennink W. E., and van Nostrum C. F. (2002). Novel crosslinking methods to design hydrogels Advanced Drug Delivery Reviews. Vol. 54 (1), 13-36.

Ho H.-S., Hsiao C.-C., Sokoloski T. D., Chen C.-Y. and Sheu M.-T. (1995). Fibrin-based drug delivery system. III: The evaluation of the release of macromolecules from microbeads. J. Control. Release, 34, 65-70.

Hong P.-D. and Chou C.-M. (2000). Phase Separation and gelation behaviors in poly(vinylidene fluoride)/tetra(ethylene glycol)dymethyl ether solutions. Polymer, 41: 8311-8320.

Kavanagh G. M., Clark A. H., and Ross-Murphy S. B. (2000) Heat-Induced Gelation of Globular Proteins: 4. Gelation Kinetics of Low pH β-Lactoglobulin Gels. Langmuir, 16, 9584-9594.

Laneuville S., Paquin P., P. Turgeon, S. L. (2000). Effect of preparation conditions on the characteristic of whey protein-xanthan gum complexes. Food hydrocolloids, 4, 305-314-

Laneuville S. I., Sanchez C., Turgeon S. L. Hardy J., and Paquin P. (2005). Small-angle static light scattering study of associative phase separation kinetics in β-lactoglobulin+xanthan gum mixtures under shear. In: Food Colloids: Interactions, Microstructure and Processing, chapter 35. Dickinson E. (Ed.), RSC Books, p. 443.

Mattison K. W., Brittain I. J. and Dubin P. L. (1995). Protein-Polyelectrolyte phase boundaries. Biotechnology Progress, 11 (6): 632-637.

Mattison K. W., Wang Y., Grymonpre K. and Dubin P. L. (1999). Micro- and Macro-phase behavior in protein-polyelectrolyte complexes. Macromolecular Symposia, 104: 53-76.

Olsson C., Langton M. and Hermansson A.-M. (2002). Microstructures of b-lactoglobulin/amylopectin gels on different length scales and their significance for rheological properties, Food Hydrocolloids, Vol. 16 (2), 111-126.

Remondetto G. E. Subirade M., (2003). Molecular mechanisms of Fe²⁺-Induced β-lactoglobulin cold gelation. Biopolymers, 69: 461-469.

Renard D., Robert P., Lavenant L., Melcion D., Popineau Y., Gueguen J., Duclairoir C., Nakache E., Sanchez C., Schmitt C. (2002) Biopolymeric colloidal carriers for encapsulation or controlled release applications. International J. of Pharmaceutics, Vol. 242, no. 1-2, 163-166.

Sanchez C., Schmitt C., Babak V. G., and Hardy J. (1997). Rheology of Whey Protein Isolate-Xanthan Mixed Solutions and Gels. Effect of pH and Xanthan Concentration.

Samant S. K., Singhal R. S., Kulkarni P. R. and Rege D. V. (1993). Review, Protein-Polysaccharide Interactions: A New Approach in Food Formulations. International Journal of Food Science and Technology, 28: 547-562.

Schmitt, C., Sanchez, C., Desobry-Banon, S., and Hardy, J. (1998). Structure and technofunctional properties of protein-polysaccharide complexes: a review. Critical Reviews in Food Science and Nutrition, 38, 689-753.

Schmitt, C., Sanchez, C., Desobry-Banon, S., and Hardy, J. (1998). Structure and technofunctional properties of protein-polysaccharide complexes: a review. Critical Reviews in Food Science and Nutrition, 38, 689-753.

Stainsby, G. Proteinaceous (1980) Gelling systems and their complexes with polysaccharides. Food chem., 6, 3-14.

Subirade M., Beaulieu L., Paquin P., US/2004 0146564 A1. Published on Jul. 29, 2004. Process for making delivery matrix and uses thereof.

Tabata Y. and Ikada Y. (1998) Protein release from gelatin matrices. Advanced Drug Delivery Reviews. Vol. 31 (3-4), 287-301.

Tolstoguzov V. B. (1997). Protein-Polysaccharide Interaction. In: Food Proteins and their Applications, pp. 171-198. Damodaran S. and Paraf A. (Eds.), Marcel Dekker, Inc., New York.

Tolstoguzov V. B. (2003). Some thermodynamic considerations in food formulation, Food Hydrocolloids, 17: 1-23.

Turgeon S. L., Beaulieu M., Schmitt C. and Sanchez C. (2003). Protein-polysaccharide interactions: phase-ordering kinetics, thermodynamic and structural aspects. Current Opinion in Colloid and Interface Science, 8 (4-5): 401-414.

Turgeon S. L. & Beaulieu M. (2001). Improvement and modification of whey protein gel texture using polysaccharides, Food Hydrocolloids, 15 (4-6): 583-591.

Veerman C. Baptist H., Sagis L., M. C., Van Der Linden E. (2003) Immune multi step Ca²⁺ induced cold gelation process for b-lactoglobuline. Journal of Agricultural and Food Chemistry. 51: 3880-3885.

Weinbreck F., de Vries R., Schrooyen P. and de Kruif C. G. (2003a). Complex Coacervation of Whey Proteins and Gum Arabic. Biomacromolecules, 4 (2): 293-303.

Zentner G. M., Bark J-S, Liu F. US 2003/0124189 A1. Published on Jul. 3, 2003. Polymer blends that swell in an acidic environment and de-swell in a basic environment.

1. A method for gelation of an undenatured globular protein and a polysaccharide, said method comprising the steps of:
a—providing a mixture of a dispersion of an undenatured globular protein and a dispersion of a polysaccharide;
b—stirring the mixture to obtain a homogeneous mixture;
c—gradually modifying the electronic charge of said undenatured globular protein and/or said polysaccharide to obtain a mixture wherein said undenatured globular 1.0 protein and said polysaccharide have opposite charges; and
d—resting the mixture of step c) for a period of time suitable to form a gel.

2. The method of claim 1, wherein gradually modifying the charges of said undenatured globular protein and said polysaccharide is achieved by gradually modifying the pH of 15 said mixture to a defined pH.

3. The method of claim 2, wherein said defined pH being lower than the isoelectric point of said undenatured globular protein when said polysaccharide is anionic.

4. The method of claim 2, wherein said defined pH being higher than the isoelectric point of said undenatured globular protein when sald polysaccharide is cationic.

5. The method according to claim 1, wherein steps a), step b), step c) and step d) take place at a temperature of about 4° C. or at room temperature.

6. The method according to claim 1, wherein the globular protein dispersion is prepared in water.

7. The method according to claim 1, wherein the polysaccharide 30 dispersion is prepared in water.

8. The method according to claim 1, wherein the globular protein and polysaccharide are mixed according to a ratio ranging from about 1:1 to about 50:1.

9. The method as claimed in any one of claim 1, wherein the polysaccharide is selected from the group consisting of polysaccharide of animal origin, polysaccharide of plant origin, polysaccharide of algal origin, polysaccharide of bacterial origin and any mixture thereof.

10. The method as claimed in claim 9, wherein the polysaccharide is selected from the group consisting of xanthan gum, gellan gum. A-carrageenan and x-carrageenan, carrageenan, alginates, pectines, carboxymethylcellulose, agar-agar, arabic gum, hyaluronate, and any mixture thereof.

11. The method as claimed in claim 1, wherein the globular protein is selected from the group consisting of BSA, ovalbumine, 8-lactoglobuiln, whey protein concentrate, whey protein isolate, soy protein and gelatin.

12. The method as claimed in claim 1, wherein the polysaccharide dispersion comprises from about 0.02 to about 5 wt % of polysaccharide.

13. The method as claimed in claims 1, wherein the globular protein dispersion comprises from about 0.02 to about 10 wt % of protein.

14. The method according to claim 1, wherein the concentration of total globular protein and polysaccharide is from about 0.02 to about 10 wt %.

15. The method as claimed in claim 1, wherein the pH is modified by addition of glucono-Nactone acid or acid producing bacteria selected from the group consisting of lactic acid producing bacteria, acetic acid producing bacteria and propionic acid producing bacteria.

16. The method as claimed in claim 15, wherein the pH is modified by addition of 30 glucono-δ-factone acid.

17. The method according to claim 16, wherein the glucono-δ-lactone acid is at a concentration from about 0.01 to about 10 wt %.

18. The method as claimed in claim 1, wherein a salt is further added to the mixture.

19. The method according to claim 18, wherein the salt is selected from the group 5 consisting of NaCl, KC′, CaCl₂, MgC12 and NaNO3.

20. The method according to claim 18, wherein the salt Is NaCl.

21. The method according to claim 20, wherein the salt is at a concentration higher than 10 about OM and lower than about 0.5M.

22. A gel obtained by the method of claim 1.

23. The gel according to claim 22, consisting of an hydrogel.

24. The gel of claim 22, comprising from about 0.03 to about 10 wt % of polysaccharide and globular protein.
